(19) United States
(12) Patent Application Publication
(10) Pub. No.: US 2022/0117959 A1
(43) Pub. Date: Apr. 21, 2022

(54) LIQUID PHARMACEUTICAL COMPOSITIONS

(71) Applicant: Vici Health Sciences LLC, Elkridge, MD (US)

(72) Inventors: Anish Dhanarajan, Catonsville, MD (US); Francesca Minale, Catonsville, MD (US)

(21) Appl. No.: 17/503,126

(22) Filed: Oct. 15, 2021

Related U.S. Application Data
(60) Provisional application No. 63/093,141, filed on Oct. 16, 2020.

(51) Int. Cl. A61K 31/4985; A61K 31/428; A61K 31/277; A61K 31/12; A61K 31/137; A61K 31/46; A61P 25/28

(52) U.S. Cl. CPC A61K 31/4985; A61K 31/428; A61K 31/277; A61P 25/28; A61K 31/137; A61K 31/46; A61K 31/12

(57) ABSTRACT

The present invention is directed to liquid pharmaceutical compositions comprising an active ingredient selected from the group consisting of pramipexole, bromocriptine, entacapone, tolcapone, selegiline, benztropine and a pharmaceutically acceptable salt thereof and a liquid vehicle. The present invention is further directed to methods of treating Parkinson's disease or one or more symptoms of Parkinson's disease comprising administering a liquid pharmaceutical composition of the present invention to a subject in need thereof.

### FIELD OF THE INVENTION

The present invention is directed to liquid pharmaceutical compositions comprising an active ingredient selected from the group consisting of pramipexole, bromocriptine, entacapone, tolcapone, selegiline, benztropine and a pharmaceutically acceptable salts thereof and a liquid vehicle. The present invention is further directed to methods of treating Parkinson's disease or one or more symptoms of Parkinson's disease comprising administering a liquid pharmaceutical composition of the present invention to a subject in need thereof.

### BACKGROUND OF THE INVENTION

Parkinson's disease is a chronic and degenerative nervous system disorder that causes loss of control of body function and movement. At least one million people in the United States, and more than five million people worldwide, have been diagnosed with Parkinson's disease. This includes about 1 in 100 people over the age of 60.

Parkinson's disease has a myriad of symptoms and complications. Symptoms of Parkinson's disease include tremors, bradykinesia, rigid muscles, impaired posture and balance, loss of automatic movements, speech changes and writing changes. Complications of Parkinson's disease include difficulty thinking, depression, difficulty in swallowing, chewing and eating, sleep disorders, bladder issues and constipation.

Several medications have been developed for the treatment of Parkinson's disease and its symptoms. These medications include levodopa, carbidopa, safinamide, ropinirole, pramipexole, bromocriptine, rotigotine, amantadine, trihexyphenidyl, benztropine, selegiline, rasagiline, tolcapone and entacapone. The majority of these medications, if not all, are available only in a solid tablet form.

Pramipexole is a partial/full agonist of several dopamine receptors including D_{2S}, D_{2L}, D₃ and D₄. Pramipexole is approved for the treatment of Parkinson's disease. Pramipexole is only available in tablet form as a hydrochloride salt in Mirapex® (Mirapex is a registered trademark of and available from Boehringer Ingelheim Pharma GMBH & Co.). Mirapex® is available in 0.125, 0.25, 0.5 1 and 1.5 milligram oral tablets and also in 0.375, 0.75, 1.5 2.25, 3, 3.75 and 4.5 milligram extended release oral tablets.

Bromocriptine is an ergoline derivative and dopamine agonist approved for the treatment of Parkinson's disease. Bromocriptine is only available in tablet or capsule form as a mesylate salt in Parlodel® or Cycloset® (Parlodel is a registered trademark of and available from Novartis AG Corporation; and Cycloset is a registered trademark of and available from Veroscience LLC.). Parlodel® is available in 2.5 milligram oral tablets and 5 milligram oral capsules. Cycloset® is available in 0.8 milligram oral tablets.

Entacapone is a selective and reversible inhibitor of the enzyme catechol-O-methyltransferase (“COMT”) approved for the treatment of Parkinson's disease. Entacapone, as the single active ingredient, is only available in tablet form in Comtan® (Comtan is a registered trademark of and available from Orion Corporation). Comtan® is available in 200 milligram oral tablets.

Tolcapone is a selective and reversible inhibitor of COMT approved for the treatment of Parkinson's disease. Tolcapone is only available in tablet form in Tasmar® (Tasmar is a registered trademark of and available from Bausch Health US, LLC). Tasmar® is available in 100 and 200 milligram oral tablets.

Selegiline, also known as L-deprenyl, is a monoamine oxidase inhibitor approved for the treatment of Parkinson's disease. Selegiline is only available as a hydrochloride salt in either tablet form in Zelapar® or capsule form in Eldepryl® or as a base in a transdermal film in Emsam® (Zelapar is a registered trademark of and available from Bausch Health US, LLC; and Eldepryl® and Emsam® are a registered trademarks of and available from Somerset Pharmaceuticals, Inc.) Zelapar® is available in 1.25 milligram orally disintegrating tablets. Eldepryl® is available in 5 milligram oral tablets. Emsam® is available in 6, 9 or 12 milligram per 24 hour extended release transdermal films.

Benztropine, also spelled benzatropine, is a selective M1 muscarinic acetylcholine receptor antagonist approved for the treatment of symptoms of Parkinson's disease. Benztropine is available as a mesylate salt in tablet or injectable form in Cogentin® (Cogentin is a registered trademark of and available from Merck Sharp & Dohme Corp). Cogentin® is available in 0.5, 1 or 2 milligram oral tablets and 1 milligram per milliliter injections.

Dysphagia, or trouble swallowing, occurs in 91 to 94% of stage 2 and stage 3 Parkinson's disease patients. Nilsson H et al., Quantitative Assessment of Oral and Pharyngeal Function in Parkinson's Disease.” Dysphagia 11: 144-150, 1996. Because of the dysphagia in Parkinson's disease patients administering solid oral medication is problematic. Healthcare providers report that administering Parkinson's disease medications in tablet form is difficult and uncomfortable for both the provider and the patient and can lead to compliance issues.

A common method for administering solid dosage forms to patients with dysphagia is to crush the solid dosage form and suspend the resulting powder in water. However, this method is highly problematic as many active ingredients are light and or water sensitive and thus degrade upon crushing and or suspension in water. Bowman C. Administration of drugs to patients with swallowing difficulties. Journal of the Malta College of Pharmacy Practice 12: 42-45, 2007. Further, many patients with dysphagia are fed through feeding tubes. These crushed tablets in water are the most common feeding tube obstruction. Bemt P, et al. Quality Improvement of Oral Medication Administration in Patients with Enteral Feeding Tubes. Quality and Safety in Health Care 2006:15: 44-47.

Another method to overcome issues with dysphagia is injections. However, injections are painful and can sometimes lead to necrosis or other injection site issues. Further, patient compliance with injections can be problematic and requires special certifications for the administrator.

Thus, there exists a need in the art, for dosage forms of medications to treat Parkinson's disease that are easy to administer. Specifically, there is a need in the art for stable liquid oral compositions.

### SUMMARY OF THE INVENTION

The present invention is directed to liquid pharmaceutical compositions comprising an active ingredient selected from the group consisting of pramipexole, bromocriptine, entacapone, tolcapone, selegiline, benztropine and a pharmaceutically acceptable salt thereof and a liquid vehicle comprising one or more carriers and one or more buffers.

The present invention is further directed to methods of treating Parkinson's disease or one or more symptoms of Parkinson's disease comprising administering a liquid pharmaceutical composition of the present invention to a subject in need thereof.

### DETAILED DESCRIPTION OF THE INVENTION

The Applicant has discovered liquid compositions of pramipexole, bromocriptine, entacapone, tolcapone, selegiline and benztropine that are surprisingly stable. The stability of these liquid compositions of the present invention is unexpected in light of the prior art, which is devoid of stable liquid compositions of pramipexole, bromocriptine, entacapone, tolcapone, selegiline or benztropine.

In one embodiment, the present invention is directed to liquid pharmaceutical compositions comprising an active ingredient selected from the group consisting of pramipexole, bromocriptine, entacapone, tolcapone, selegiline, benztropine and a pharmaceutically acceptable salt thereof and a liquid vehicle comprising one or more carriers and one or more buffers.

In another preferred embodiment, the pramipexole, bromocriptine, entacapone, tolcapone, selegiline, benztropine or a pharmaceutically acceptable salt thereof is present in compositions of the present invention at a concentration from about 0.0001% to about 20% w/v and preferably from about 0.02% to about 10% w/v.

In another preferred embodiment, pramipexole or a pharmaceutically acceptable salt thereof is present in compositions of the present invention at a concentration from about 0.0001% to about 1% w/v and more preferably from about 0.002% to about 0.1% w/v.

In another preferred embodiment, bromocriptine or a pharmaceutically acceptable salt thereof is present in compositions of the present invention at a concentration from about 0.001% to about 1% w/v and more preferably from about 0.02% to about 0.2% w/v.

In another preferred embodiment, entacapone or a pharmaceutically acceptable salt thereof is present in compositions of the present invention at a concentration from about 0.1% to about 20% w/v and more preferably from about 2% to about 10% w/v.

In another preferred embodiment, tolcapone or a pharmaceutically acceptable salt thereof is present in compositions of the present invention at a concentration from about 0.1% to about 20% w/v and more preferably from about 1% to about 10% w/v.

In another preferred embodiment, selegiline or a pharmaceutically acceptable salt thereof is present in compositions of the present invention at a concentration from about 0.001% to about 1% w/v and more preferably from about 0.04% to about 0.2% w/v.

In another preferred embodiment, benztropine or a pharmaceutically acceptable salt thereof is present in compositions of the present invention at a concentration from about 0.0001% to about 1% w/v and more preferably from about 0.005% to about 0.1% w/v.

In another preferred embodiment, the one or more carriers are selected from the group consisting of water, propylene glycol and glycerin, preferably a mixture of water and glycerin.

In another preferred embodiment, the one or more carriers are present in compositions of the present invention at a concentration from about 70% to about 120% w/v, more preferably from about 90% to about 115% w/v and even more preferably from about 95% to about 113% w/v.

In a more preferred embodiment, water is present in compositions of the present invention at a concentration from about 10% to about 99.999% w/v, more preferably from about 10% to about 90% w/v, even more preferably from about 20% to about 70% w/v.

In another more preferred embodiment, glycerin is present in compositions of the present invention at a concentration from about 10% to about 99.999% w/v, more preferably from about 10% to about 90% w/v, even more preferably from about 40% to about 80% w/v, yet even more preferably from about 50% to about 70% w/v and most preferably at about 60% w/v.

In another more preferred embodiment, propylene glycol is present in compositions of the present invention at a concentration from about 10% to about 99.999% w/v, more preferably from about 10% to about 90% w/v and even more preferably from about 20% to about 60% w/v.

In a yet more preferred embodiment the one or more carriers are selected from: a mixture of from about 10% to about 90% w/v water and from about 10% to about 90% w/v glycerin; a mixture of from about 10% to about 90% w/v water and from about 10% to about 90% w/v propylene glycol; a mixture of from about 40% to about 60% w/v water and from about 50% to about 70% w/v glycerin; and a mixture of from about 50% to about 70% w/v water and from about 30% to about 50% w/v propylene glycol.

In another preferred embodiment, the one or more buffers are selected from the group consisting of acetate buffers, carbonate buffers, citrate buffers including citric acid and a citrate salt, phosphate buffers and borate buffers. In a preferred embodiment, the one or more buffers is a mixture of citric acid and a citrate salt.

Citrate salts include, but are not limited to, salts that pair a cation with the up to three carboxylate ions that can form from deprotonating the three carboxylic acid groups of citric acid. For example, a salt of sodium citrate may be formed by replacing one, two, or three of the carboxylic acid protons with sodium ions (i.e., monosodium citrate, disodium citrate, and trisodium citrate). The citrate salts may be added to compositions of the present invention as part of an aqueous solution, or as a solid. When added as solid, the citrate compound may be anhydrous, or more typically a hydrate that incorporates one or more water (“H₂O”) group into the crystal structure of the compound. For example, solid sodium citrate may incorporate one or more water groups into the crystal structure, such as sodium citrate monohydrate (i.e., 1H₂O), sodium citrate dihydrate (i.e., 2H₂O), sodium citrate trihydrate (i.e., 3H₂O), sodium citrate tetrahydrate (i.e., 4H₂O), sodium citrate pentahydrate (i.e., 5H₂O), sodium citrate hexahydrate (i.e., 6H₂O), etc. Citrate salts may also include the hydrates and/or anhydrates of salts beyond sodium, such as other alkali metal or alkaline metal cations, ammonia, organic primary, secondary, or tertiary amines including, but not limited to; lithium, potassium, calcium, magnesium and aluminum cations and the like; nontoxic quaternary ammonia and amine cations including ammonium, tetramethylammonium, tetraethylammonium, methylammonium, dimethylammonium, trimethylammonium, triethylammonium, diethylammonium, and ethylammonium and the like; and organic amines including ethylenediamine, ethanolamine, diethanolamine, piperidine, piperazine and the like. In a preferred embodiment, the citrate salt is a sodium citrate. In a more preferred embodiment, the sodium salt is a trisodium citrate. In an even more preferred embodiment, the citrate salt is a sodium citrate dihydrate or a trisodium citrate dihydrate.

In a more preferred embodiment, the one or more buffers is present in compositions of the present invention at a concentration from about 0.01% to about 2.0% w/v, more preferably from about 0.1% to about 1.5% w/v and most preferably at about 0.22% w/v or about 1.02% w/v.

In another more preferred embodiment, citric acid is present in compositions of the present invention at a concentration from about 0.01% to about 1.0% w/v and more preferably from about 0.1% to about 0.3% w/v or from about 0.15% to about 0.45% w/v.

In another more preferred embodiment, the citrate salt is present in compositions of the present invention at a concentration from about 0.01% to about 2.0% w/v and more preferably from about 0.02% to about 0.08% w/v or from about 0.5% to about 1.0% w/v.

In a preferred embodiment liquid pharmaceutical compositions of the present invention may have a pH from about 2 to about 10, preferably from about 2 to about 7, more preferably from about 3 to about 6.

In another embodiment, liquid pharmaceutical compositions of the present invention may contain an antimicrobial agent. Antimicrobial agents suitable for use in the present invention include, but are not limited to, benzyl alcohol, benzalkonium chloride, parabens including methylparaben, propylparaben, chlorobutanol, edetate, citrate, pentetate, tromethamine, sorbate, ascorbate, sodium benzoate, sulfites containing agents and mixtures thereof. In a preferred embodiment, the antimicrobial agent is selected from the group consisting of methylparaben, propylparaben, sodium benzoate and a combination thereof.

In a more preferred embodiment, the antimicrobial agent is present in compositions of the present invention at a concentration from about 0.01% to about 1.0% w/v and more preferably from about 0.05% to about 0.5% w/v.

In another preferred embodiment, the liquid pharmaceutical compositions of present invention do not contain an antioxidant.

In another preferred embodiment, the liquid pharmaceutical compositions of present invention may contain an antioxidant. Antioxidants suitable for use in the present invention include, but are not limited to, butylated hydroxyanisole (“BHA”), butylated hydroxytoluene (BHT), methionine, sodium ascorbate, sodium thiosulfate, thioglycerol, ascorbic acid, ascorbyl palmitate, propyl gallate, dL-alpha-tocopherol, sodium sulfite, sodium metabisulfite, sodium bisulfite cysteine hydrochloride, glutathione and a mixture thereof. In a preferred embodiment, the antioxidant is BHA.

In a more preferred embodiment, when antioxidants are present in compositions of the present invention the antioxidants are present at a concentration from about from about 0.001% to about 1.0% w/v and preferably from about 0.005% to about 0.1% w/v.

In another preferred embodiment, liquid pharmaceutical compositions of the present invention may contain a flavoring agent. Flavoring agents suitable for the present invention include, but are not limited to, peppermint oil, menthol, spearmint oil, citrus oil, cinnamon oil, strawberry flavor, cherry flavor, raspberry flavor, orange oil and a combination thereof. In a preferred embodiment, the flavoring agent is strawberry flavor.

In a more preferred embodiment, the flavoring agent is present in compositions of the present invention at a concentration from about 0.001% to about 0.1% w/v and more preferably from about 0.005% to about 0.05% w/v.

In another preferred embodiment, liquid pharmaceutical compositions of the present invention may contain a sweetener. Sweeteners suitable for use in the present invention include, but are not limited to, sucralose, sucrose, aspartame, saccharin, dextrose, mannitol, glycerin, xylitol and combinations thereof. In a preferred embodiment, the sweetener is sucralose.

In a more preferred embodiment, the sweetener is present in compositions of the present invention at a concentration from about 0.0001% to about 0.01% w/v and more preferably from about 0.001% to about 0.005% w/v.

In another preferred embodiment, the compositions of the present invention provide stability of the active ingredient. Preferably the active ingredients of the compositions of the present invention maintain at least 90% initial assay value for one week at 40° C., more preferably at least 90% initial assay value for two weeks at 40° C. and even more preferably at least 90% initial assay value for four weeks at 40° C. Further and preferably the compositions of the present invention contain less than 4% w/v total impurities following incubation for 144 hours at 60° C., more preferably less than 3% w/v total impurities following incubation for 144 hours at 60° C., even more preferably less than 2% w/v total impurities following incubation for 144 hours at 60° C. and most preferably less than 1% w/v total impurities following incubation for 144 hours at 60° C.

In another embodiment, the present invention is directed to methods of treating Parkinson's disease comprising administering an effective amount of a liquid pharmaceutical composition of the present invention to a subject in need thereof.

In another embodiment, the present invention is directed to methods of treating one or more symptoms of Parkinson's disease comprising administering an effective amount of a liquid pharmaceutical composition of the present invention to a subject in need thereof.

In a preferred embodiment, administration of the liquid pharmaceutical compositions of the present invention occur via the oral route.

In another preferred embodiment, administration of the liquid pharmaceutical compositions of the present invention occurs via a feeding tube.

In another embodiment, the present invention is directed to methods for detecting the presence of an active ingredient selected from the group consisting of rasagiline, ropinirole or a pharmaceutically acceptable salt thereof in a fluid sample comprising:
a) providing a detecting agent;
b) contacting the detecting agent with the fluid sample; and
c) determining the presence of the active ingredient bound to the detecting agent.

In a preferred embodiment, the presence of the active ingredient bound to the detecting agent is determined by fluorescence.

In another embodiment, the present invention is directed to methods for determining purity of an active compound selected from the group consisting of rasagiline, ropinirole or a pharmaceutically acceptable salt thereof in a fluid sample comprising:
a) dissolving the fluid sample in a first solvent to produce a sample solution;
b) dissolving a pure sample of the active compound in a second solvent to produce a reference solution;
c) subjecting the sample solution to a chromatic technique comprising a stationary phase;
d) subjecting the reference solution to the chromatic technique; and
e) comparing the results of c) and d) to determine the presence of one or more related substances in the sample solution.

In a preferred embodiment, the first solvent and the second solvent are each independently selected from the group consisting of an aqueous buffer, an organic solvent and a combination thereof.

In another preferred embodiment, the stationary phase is selected from the group consisting of a reverse phase (hydrophobic), octylsilyl silica gel, octadecylsilyl silica gel, phenyl gel and a combination thereof.

In another preferred embodiment, the liquid pharmaceutical compositions of the present invention containing pramipexole, bromocriptine, entacapone, tolcapone, selegiline, benztropine or a pharmaceutically acceptable salt thereof are administered at an amount of from about 0.1 to about 1,000 milligrams, more preferably from about 0.1 to about 200 milligrams.

In a more preferred embodiment, the liquid pharmaceutical compositions of the present invention containing pramipexole or a pharmaceutically acceptable salt thereof are administered at an amount of from about 0.125 to about 4.5 milligrams, more preferably from about 0.125 to about 1.5 and even more preferably at about 0.125, 0.25, 0.375, 0.5, 0.75, 1.0, 1.5, 2.25, 3.0, 3.75 or 4.5 milligrams.

In a more preferred embodiment, the liquid pharmaceutical compositions of the present invention containing bromocriptine or a pharmaceutically acceptable salt thereof are administered at an amount of from about 0.8 to about 5 milligrams and more preferably at about 0.8, 2.5 or 5 milligrams.

In a more preferred embodiment, the liquid pharmaceutical compositions of the present invention containing entacapone or a pharmaceutically acceptable salt thereof are administered at an amount of about 200 milligrams.

In a more preferred embodiment, the liquid pharmaceutical compositions of the present invention containing tolcapone or a pharmaceutically acceptable salt thereof are administered at an amount of from about 100 to about 200 milligrams and more preferably at about 100 or 200 milligrams.

In a more preferred embodiment, the liquid pharmaceutical compositions of the present invention containing selegiline or a pharmaceutically acceptable salt thereof are administered at an amount of from about 1.25 to about 5 milligrams and more preferably at about 1.25 or 5 milligrams.

In a more preferred embodiment, the liquid pharmaceutical compositions of the present invention containing benztropine or a pharmaceutically acceptable salt thereof are administered at an amount of from about 0.5 to about 2 milligrams and more preferably at about 0.5, 1 or 2 milligrams.

As used herein the term “pharmaceutically acceptable” refers to ingredients that are not biologically or otherwise undesirable in an oral application.

As used herein, all numerical values relating to amounts, weights, and the like, are defined as “about” each particular value, that is, plus or minus 10%. For example, the phrase “10% w/w” is to be understood as “9% to 11% w/w.” Therefore, amounts within 10% of the claimed value are encompassed by the scope of the claims.

As used herein “% w/w” refers to the weight percent by weight of the total formulation.

As used herein “% w/v” refers to the weight percent by volume of the total formulation.

As used herein the term “effective amount” refers to the amount necessary to treat a patient in need thereof.

As used herein the term “treatment” or “treating” refers to alleviating or ameliorating Parkinson's disease or symptoms of Parkinson's disease.

As used herein, the term “stable” includes, but is not limited to, physical and chemical stability.

Pharmaceutically acceptable salts of that can be used in accordance with the current invention include but are not limited to hydrochloride, dihydrate hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, mesylate, maleate, gentisinate, fumarate, tannate, sulphate, tosylate, esylate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts.

Throughout the application, the singular forms “a,” “an,” and “the” include plural reference unless the context clearly dictates otherwise.

Throughout the application, all disclosed ranges include all possible values within those ranges. All possible values within the ranges disclosed in the application can also be used as endpoints for additional ranges between these values.

The disclosed embodiments are simply exemplary embodiments of the inventive concepts disclosed herein and should not be considered as limiting unless the claims expressly state otherwise.

The following example is intended to illustrate the present invention and to teach one of ordinary skill in the art how to use the formulations of the invention. The example is not intended to be limiting in any way.

### EXAMPLE

### Example 1—Active Ingredient Vehicle Stability Screening (Prophetic)

### Method

Pramipexole, bromocriptine, entacapone, tolcapone, selegiline and benztropine were each added separately to various combinations of excipients. Each formulation was then stored at greater than room temperatures (e.g. about 26 to about 75 degrees Celsius) for an extended amount of time (e.g. about 1 week to about 1 year). Assay and degradants were recorded at several time points throughout the storage.

### Results

Each of the formulations provided stable active ingredient assays upon storage.

What is claimed is:

1. A liquid pharmaceutical composition comprising an active ingredient selected from the group consisting of pramipexole, bromocriptine, entacapone, tolcapone, selegiline, benztropine and a pharmaceutically acceptable salt thereof and a liquid vehicle comprising one or more carriers and one or more buffers.

2. The composition of claim 1, wherein the one or more carriers are selected from the group consisting of water, propylene glycol and glycerin.

3. The composition of claim 1, wherein the one or more buffers are selected from an acetate buffer, a carbonate buffer, a citrate buffer, a phosphate buffer and a borate buffer.

4. The composition of claim 1, further comprising an antimicrobial agent.

5. The composition of claim 4, wherein the antimicrobial agent is selected from the group consisting of sodium benzoate, parabens and mixtures thereof.

6. A method of treating Parkinson's disease comprising administering an effective amount of the composition of claim 1 to a subject in need thereof.

7. A method of treating one or more symptoms of Parkinson's disease comprising administering an effective amount of the composition of claim 1 to a subject in need thereof.
